(19) United States
(12) Patent Application Publication
(10) Pub. No.: US 2010/0094383 A1
(43) Pub. Date: Apr. 15, 2010

(54) IRRADIATION DEVICE BY MODULATED PHOTONIC ENERGY RADIATION

(76) Inventors: Jean-Pierre Breda, La Ravoire (FR); Eric D. Cordemans De Meulenaer, Wezembeek (BE)

Correspondence Address: OLIFF & BERRIDGE, PLC, P.O. BOX 320850, ALEXANDRIA, VA, 22320-4850, US

(21) Appl. No.: 12/449,804

(22) PCT Filed: Mar. 20, 2008
(86) PCT No.: PCT/FR2008/000372

§ 371 Date: Aug. 27, 2009

(30) Foreign Application Priority Data
Mar. 21, 2007 (FR) 0702068

(51) Int. Cl. A61N 5/06; G21K 5/00; C12N 13/00

(52) U.S. Cl. 607/88; 250/492.1; 435/173.8; 435/173.1

(57) ABSTRACT

An irradiation device by modulated photonic energy radiation comprising a photonic emitter module (11) emitting nanometric-frequencies. The emitter module is connected to modulating means performing a first chopping at low frequency (LF) and a second chopping at radiofrequency (RF) to obtain a sequence of pulses at low frequency (LF) chopped at radiofrequency (RF).

### BACKGROUND OF THE INVENTION

The invention relates to an irradiation device by modulated photonic energy radiation comprising a photonic emitter module emitting at nanometric-frequency photons, of predefined wavelength.

This type of irradiation device uses a light-emitting diode (LED) or laser emitter module for photo bio stimulation (PBS) applications, in particular to stimulate or modify a defense reaction of the body cells, in particular achieving great relief in case of pain of inflammatory and physiological origin.

Living cells react in different ways to electromagnetic, chemical, thermal or mechanical stresses.

Proteomic analysis has enabled sequences of proteins involved in stress and defense mechanisms and in response reactions to light to be identified. In spite of the complexity due to the set of participants involved in the implemented sequence, such reaction mechanisms are quick (about a few minutes).

It is now acquired that specific combinations of reaction paths, often interacting with one another, lead to distinct modes of transcription in the course of stress phenomena (stress is defined as conditions of application of energies to the living medium that are not those encountered by the cells in a usual physiological manner). Application of a soft energy stress enables both the physiological cell mechanisms to be modified, but also, through the envisaged application, enables their kinetics to be influenced and oriented towards the goal sought for.

In the case of plants for example, this regulating network enables very finely adapted responses to be provided to the different stimuli which manage the development of the cells, tissues and organs.

Most of the protein sequences generated by stress conditions at the same time involve defense reactions. Similar sequences are involved in the same stress—defense mechanisms in animal cells and yeasts.

Physiological mechanisms (protein synthesis, stimulation and modification of endogenous metabolite synthesis mechanisms, modification of the sigma factors, implication of different genetic sequences, poration, etc. . . . ) modified by application of this stress can, depending on the energies involved, be temporary and/or reversible, or result in selective destruction of certain populations, whereas other populations are modified differently, resulting in growth or decrease thereof.

Cell responses further depend on other factors such as the irradiation frequency, power and duration, and the nature of the coupling media between the emitting module and the treated medium.

By an original combination of three electromagnetic wave fields, the present technology enables the effects to be directed according to the frequency ranges used and the parameters on which they depend.

### STATE OF THE ART

The document U.S. Pat. No. 6,602,275 describes a phototherapy device making use of three series of LEDs of different wavelengths (470 nm, 630 nm, and 880 nm). A control circuit is connected between the power supply and the LEDs, and comprises an electronic processor connected by means of switches and a digital-to-analog converter to operational amplifiers designed to control FET transistors associated with the different series of LEDs. The system enables adjustment of the power level, the duration and frequency of repetition and sequence of the power supply pulses.

Documents U.S. Pat. No. 3,280,816, FR 1342772 and FR 1501984 concern electron emission in a broad 15 MHz to 300 MHz frequency spectrum, requiring a huge amount of energy (to achieve a plasma) of hyper-frequencies and HF frequencies. At these frequencies and these powers, the thermal effect is great.

The U.S. Pat. No. 5,584,863 refers to apparatus for electromagnetic treatment which uses high frequencies, at high energies, but without any thermal effect.

U.S. Pat. No. 5,800,481 describes the use of two low-frequency oscillators connected on input of a logic AND circuit, the first oscillator operating at 2.4 Hz and the second operating at 0.5 Hz. The use of these frequencies enables the cortical processes to be stimulated and acts on the nervous system.

Photo bio stimulation (PBS) in the red to near infrared range (630-1000 nm) accelerates wound healing, improves recovery from ischemic cardiac injuries and attenuates degeneracy of an injured optic nerve (see US Patent 2004215293).

LED phototherapy has also been used to speed up hatching and to reduce the mortality rate of chickens' eggs.

### OBJECT OF THE INVENTION

The object of the invention relates to an irradiation device enabling enhanced penetration of electromagnetic radiation into a medium to be treated by optimally stimulating the physiological reactions of the cells of the organism.

The irradiation device by photonic energy radiation comprises a nanometric-frequency photonic emitter module and is characterized in that the emitter module is connected to modulating means performing a first low-frequency LF chopping and a second radiofrequency RF chopping to obtain a sequence of low-frequency LF pulses chopped at radiofrequency RF.

The object of the first chopping at low frequency LF is stimulation of the cells, the object of the second chopping at radiofrequency RF being depolarization of the cells.

According to a development of the invention, the low frequency LF chopping has a frequency range comprised between 5 Hz and 50 Hz and preferably between 10 Hz and 20 Hz. The radiofrequency RF chopping has a frequency range comprised between 500 kHz and 10,000 kHz and preferably between 1,000 kHz and 1,500 kHz.

According to a development of the invention, the photonic emitter module, of preset wavelength, is connected to a DC current source by a transistor to form a chopping circuit.

The chopping circuit can comprise a logic AND circuit, a first low-frequency oscillator and a second radiofrequency oscillator being connected to the inputs of this circuit, the output of the logic AND circuit being connected to the transistor gate to modulate the power supply with double-chopping by the two frequencies of said first and second oscillators.

According to a development, the emitter module is equipped with a series or light-emitting diodes (LEDs) or laser diodes with radiation near the infrared, and of GaAlAs type.

The irradiation process using the device implements original combination of three electromagnetic wave fields, i.e.:
nanometric-frequency photon emission,
chopping of the nanometric emission by low frequency to stimulate the cells and enable a more efficient action of the nanometric waves,
chopping of the nanometric emission by radiofrequency to depolarize the cells thereby enabling more efficient penetration of the photons.

Radiation of the nanometric-frequency photons modulated by LF and RF is emitted by light-emitting diodes (LEDs) or laser diodes. The resulting signal is made of a photon emission by electromagnetic waves of several hundreds of THz (TeraHertz=10¹² Hz) broken down into pulses by a low frequency and a radiofrequency and modulated by the harmonics of these two frequencies.

This photon radiation is performed under soft energy conditions.

It is the combination of chopping and nanometric frequency modulation by two frequencies and modulation by their harmonics that causes the effect sought for. Propagation of these waves takes place without any direct contact with the target that living organisms represent, under soft energy conditions proper to the fundamental mechanisms of life and without addition or use of any agent or reagent of chemical or other nature. The advantage of this technique lies in the fact that it enables easier penetration of the wave, while at the same time only using a low energy.

The effects observed by application of the method according to the invention are among others:
modification of germination of seeds, of emergence of seedlings and of plant growth,
physiological effects of relief from muscular pains, sprains, backache, and post-traumatic pains.

### 1) Physical Effects of Wave Penetration: the Physical Effect is Due to the RF Modulation.

Our choice involved an egg as being representative of a living medium, and infrared irradiation close to 875 nanometers.

The IR emitter delivers 100 mW/cm², and is placed 20 cm from the egg. Measurement is made at the center of the egg by means of an IR sensor.

| | Measurement | Attenuation | IR wave |
|---|---|---|---|
| Treatment | (mW/cm²) | factor | penetration effect |
| IR only | 0.5 | 200 | Weak penetration |
| IR with LF | 0.5 | 200 | Weak penetration |
| modulation | | | |
| IR with RF | 10 | 10 | RF is the IR carrier |
| modulation | | | |
| IR with LF + RF | 10 | 10 | RF performs |
| modulation | | | penetration, LF the |
| | | | biocellular effect |

### 2) Biocellular Response Effects: the Effect is Due to the Low Frequencies (LF).

a) treatment of radish seeds:

Germination tests after exposure of the seeds for 15 minutes, then irradiation for 15 min/day after planting.

| Treatment | Germination | Growth |
|---|---|---|
| Control | 3 days | |
| IR only | 3 days | Identical to control |
| IR modulation | 5 days | Late development (caught up with respect |
| (LF + RF) | | to the others after 3-4 days (D0 + 8-9: |
| | | therefore faster growth on outcome. |
| | | The plant is more vigorous, the leaves are |
| | | already formed and greener after 8 days |
| | | (D0 + 14). |

b) treatment of cucumber seeds:

No irradiation before planting, but irradiation of the pots every day 15′/day after planting.

| Treatment | Germination | Growth |
|---|---|---|
| Control | 8 days | |
| IR only | 8 days | Faster growth than control |
| IR modulation | 6 days | Faster growth, but also more vigorous |
| (LF + RF) | | growth (greener leaves) |

c) treatment of lettuce and parsley seeds

Parsley seeds are difficult to make germinate (it generally takes 40 days to get them to germinate).

Results after 21 days:

### Germination

| Treatment | Parsley germination | Lettuce germination |
|---|---|---|
| IR only | No germination | Little germination |
| Control | No germination | Medium germination |
| IR modulation | Very numerous germination | Very numerous germination |
| (LF + RF) | | |

15′ irradiation before planting, but irradiation of the pots every day 15′/day after planting.

The effects on the parsley and lettuce seeds are different:

Pulsed irradiation makes the seeds germinate more.

Continuous irradiation makes lettuce seeds germinate less, but those that have sprouted have a higher uptake than those that are pulse irradiated. For parsley on the other hand, which is known to be difficult to grow, after a long delay, only pulsed irradiation enables uptake. Neither continuous irradiation or natural sowing enable fast germination.

### Growth

| Treatment | Parsley growth | Lettuce growth |
|---|---|---|
| IR only | — | More vigorous uptake |
| Control | — | Medium uptake |
| IR modulation | Uptake only under | Medium uptake |
| (LF + RF) | pulsed conditions | |

### 3) Effects on Permeabilization of the Cells by Alamar Blue Test.

Permeabilization of the cell membrane was analyzed by transfer of a molecule into the cells.

FIG. 3 illustrates an Alamar Blue test. Alamar is a compound that changes color when it is reduced inside the cell, it then changes from blue to red and becoming fluorescent, making reading of the fluorescence emitted by a fluorimeter possible. The graph of FIG. 3 corresponds to an Alamar Blue test on human thyrocytes comparing the double-pulsed IR mode (modules at 12 Hz and 1,200 kHz) with continuous IR mode. Luminescence analysis is performed during 15 minutes of irradiation, and again after irradiation has been stopped.

Pulsed mode shows that the transmembrane traffic is very high as soon as irradiation is stopped.

FIG. 4 illustrates the effects on reduction of the Alamar Blue in pig thyrocytes, only the LF+HF group corresponding to pulsed mode of the I.R. modulated at low frequency (12 Hz) and at radiofrequency (1,200 kHz) is significantly different.

### 4) Transepithelial Resistance (TER or TEER) Modification Measurement.

This measurement indicates the permeability rate between adjacent cells, illustrating the possibility of molecules passing from one cell to the other.

FIG. 5 illustrates the effects of pig thyrocytes cultivated in a bicameral system on TER. Ctrl corresponds to measurement of the non-irradiated cells, LF corresponds to I.R. irradiation modulated at low frequency (12 Hz), HF corresponds to I.R. irradiation modulated at radiofrequency (1,200 kHz), and LF+RF corresponds to double-modulated I.R. irradiation (12 Hz and 1,200 kHz). The measurements are made during 15 minutes of irradiation, and then after irradiation has ceased.

The results show that only pulsed mode, during irradiation itself, deactivates the cell membrane barrier functions during irradiation (the transepithelial resistance corresponds to another view of the permeability of the membrane, therefore the higher the permeability, the lower the TER). As soon as irradiation is ceased, the barrier functions between the cells are reactivated, each cell resuming its autonomy and internal dynamism. Return to normal physiological conditions takes place extremely quickly in the case of double modulation. The results indicate an action at inter-cell level, without long-duration irradiations having to be applied.

### 5) Effects on Proliferation of Rat Thyroid Epithelial Cells (Cell Line PCCL3)

Under optimal culture conditions such as those indicated by A. Fusco, M T. Berlingieri, P P Di Fiore, G. Portella, M. Grieco, and G. Vecchio (1987 One- and two-step transformations of rat thyroid epithelial cells by retroviral oncogenes. Mol Cell Biol 7: pp 3365-3370), cells that have undergone cellular stress due to freezing and thawing take from one to two weeks before recovering and then multiplying normally.

FIG. 6 illustrates the percentage of cells 24 and 48 hours after thawing of the PCCL3 cell line. The cells are irradiated for 15 minutes and then placed in culture. CW corresponds to non-modulated I.R. irradiation, HF corresponds to I.R. irradiation modulated at radiofrequency (1,200 kHz) and LF+HF corresponds to double-modulated I.R. irradiation (12 Hz and 1,200 kHz). Only the LF+HF group is significant. The decrease of the number of cells in the CW group means that this type of irradiation does not enable the cells to recover their normal vigor, and many of them that have become too fragile do not survive. Only double-modulation mode synergizes a significant proliferation, right from the moment irradiation begins. This make it possible to envisage using the present technology to rapidly initiate proliferation of lines that are traditionally fragile and difficult to cultivate.

These results indicate that double modulation induces different effects from those obtained by simple modulation at low frequencies or at radiofrequencies. We do attend to initiation of fundamental cell mechanisms.

### BRIEF DESCRIPTION OF THE DRAWINGS

Other advantages and features will become more clearly apparent from the following description of an embodiment of the invention given for non-restrictive example purposes only and represented in the accompanying drawings, in which:

FIG. 1 represents a simplified drawing of the electronic circuitry of the irradiation device according to the invention;

FIG. 2 shows the shape of the pulse train on output from the chopping circuit;

FIGS. 3 to 6 represent results relating to use of the device according to the invention.

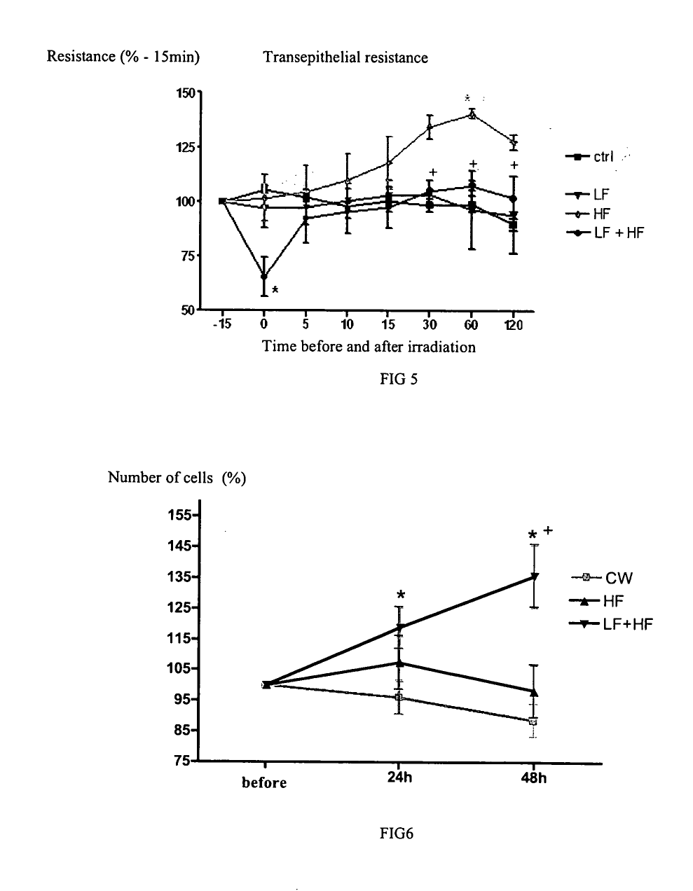

### DETAILED DESCRIPTION OF THE INVENTION

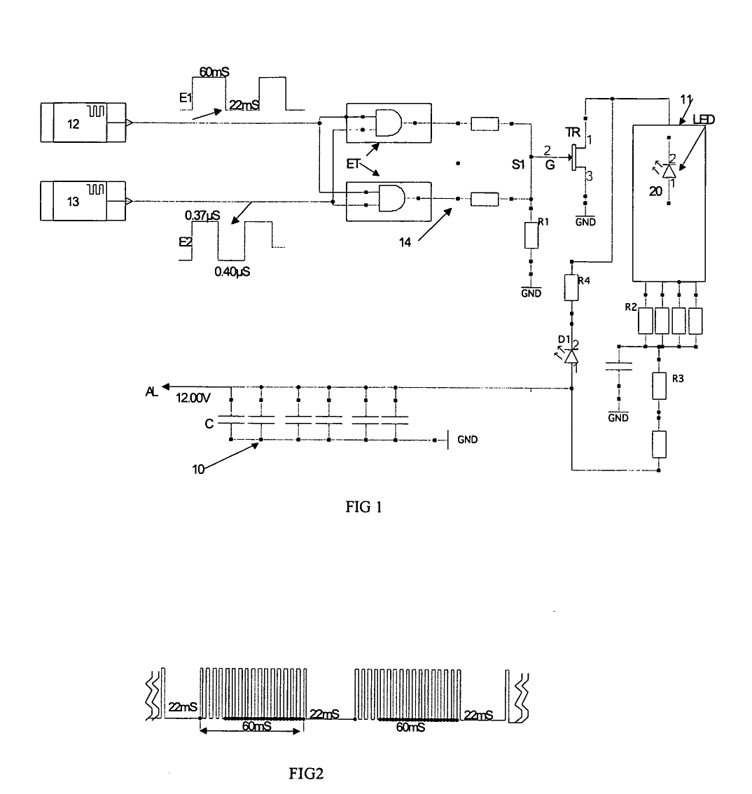

With reference to FIG. 1, an irradiation device 10 by modulated photonic energy radiation is composed of a photonic emitter module 11 able to deliver a nanometric electromagnetic wave, in particular of several hundreds of THz (TeraHertz 10¹²).

Emitter module 11 is connected to modulating means performing a first chopping at low frequency LF the frequency range whereof is preferably comprised between 5 Hz and 50 Hz or more particularly between 10 Hz and 20 Hz, and a second chopping at radiofrequency RF the frequency range whereof is preferably comprised between 500 kHz and 10,000 kHz or more particularly between 1,000 kHz and 1,500 kHz. This double modulation enables a sequence of low frequency LF pulses chopped at radiofrequency RF to be obtained.

Photonic emitter module 11 is formed by a plurality of light-emitting diodes

(LED) or laser diodes (a single one is represented in FIG. 1) with radiation near the infrared, for example of the GaAlAs type.

According to a particular embodiment, the LEDs are supplied by a DC power source AL at very low voltage (for example between 3V and 12V) via a control transistor TR. The emitter module preferably emits light energy in the ultraviolet, visible or (near or far) infrared range.

Control transistor TR can be a field-effect transistor FET whose gate is electrically connected to the output S1 of at least one logic AND circuit, the assembly forming a chopping circuit 14. Transistor TR and logic AND circuit can naturally be replaced by any other component to perform chopping of the supply pulses.

A first low-frequency oscillator 12 and a second radiofrequency oscillator 13 are connected to the input of the logic AND circuit. The output circuit of the logic AND circuit is connected to the gate of transistor TR so as to modulate the power supply with double chopping by the two frequencies LF and RF of the first and second oscillators.

When the signals from the first and second oscillators reach the respective inputs of the logic AND circuit, the output of the logic AND circuit thus delivers a series of pulses in the form of low frequency LF pulses chopped at radiofrequency RF.

For example, first 12 Hz-frequency oscillator 12 delivers square pulses E1 of 60 ms every 82 ms (Duty Cycle=73%). Second 1,230 kHz-frequency oscillator 13 delivers pulses E2 of 0.37 ms every 0.77 ms (Duty Cycle=48%).

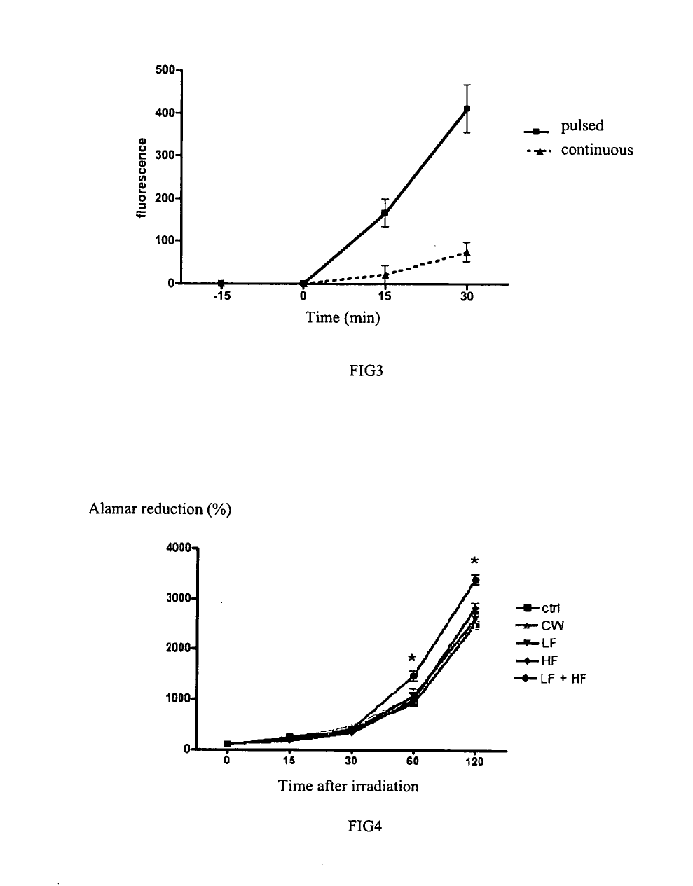

FIG. 2 illustrates the pulse train on output S1 of chopping circuit 14, i.e. a total of 77,922 pulses of 0.37 ms every 0.77 ms for 60 ms and renewed every 82 ms. The pulses are thereby generated during 60 ms, then the output of circuit S1 no longer generates anything for 22 ms before performing a new pulse cycle, etc.

These chopping pulses are applied to gate G of transistor TR, which is connected to the ground by a resistor R1. One of the electrodes of transistor TR is in contact with the ground, and the other electrode is connected to the cathode of light-emitting diodes LED. The anode of the LEDs is connected to DC source AL, either directly or via resistors R2, R3. DC source AL can be formed by a battery or an AC-DC converter associated with a low-voltage regulation circuit.

LEDs emit radiations of the same wavelengths and are advantageously connected in series in a single branch. Several branches can be connected in parallel according to the number of LEDs.

An on-off indicating diode D1 is connected in series with a resistor R4 between DC source AL and the cathode of LEDs of emitter module 11. Capacitors C are connected in parallel between source AL and ground.

The presence of chopping circuit 14 controlled by the two oscillators 12, 13 ensures pulsed power supply of LEDs for emission of the nanometric electromagnetic wave of several hundred THz, which is chopped into pulses by the 12 Hz low frequency and the 1,230 kHz radiofrequency. This double chopping generates harmonics which modulate the main emission in THz at the 12 Hz and 1,230 kHz frequencies.

The present invention is not limited to use of a chopping circuit and a logic AND gate. Double modulation by chopping at low frequency LF and at radiofrequency RF can in fact be achieved without having recourse to the use of a logic AND gate. For example the irradiation device can comprise an oscillator at radiofrequency RF turned off and on by an oscillator at low frequency LF acting on the radiofrequency RF oscillator.

According to another example, the device can comprise a radiofrequency oscillator controlled by a microcontroller itself applying low-frequency chopping by counting the pulses of the radiofrequency oscillator. Thus, referring back to the example of FIG. 2, the microcontroller enables a pulse train comprising 77,922 pulses of 0.37 ms to be obtained every 0.77 ms for 60 ms and renewed every 82 ms. The pulses are generated during 60 ms then the microcontroller stops the oscillator for 22 ms before performing a new pulse cycle, etc.

It has been observed that the use of such nanometric electromagnetic waves chopped and modulated by a low frequency and a radiofrequency also enables the cells of the organism to be stimulated, in particular providing considerable relief from pains of inflammatory and physiological origin (sprains, backache, post-traumatic pains, etc. . . . ). Chopping at low frequency (12 Hz) enhances penetration of the electromagnetic waves into the cells, and chopping at radiofrequency (1,230 kHz) depolarizes the medium to be passed through. Emission of radiation produced by the LEDs has a central frequency of about 875 nm (343 THz) which is situated at the limit of the visible and of infrared. The mean power emitted is low, about 0.3 W/cm².

The chopping frequency values can be modified depending on the effect sought to be achieved for a predefined application. The LF frequency of the first oscillator can preferably be comprised between 5 Hz and 50 Hz or more particularly between 10 Hz and 20 Hz, whereas the RF frequency of the second oscillator is comprise between 500 kHz and 10,000 kHz or more particularly between 1,000 kHz and 1,500 kHz.

The cell responses further depend on other factors, such as the irradiation frequency, power and duration of the irradiation, and the nature of the coupling media between emitter module 11 and the treated medium. The irradiation sequences and their repetition over time also play a role in modulating stimulation of the cell responses.

The LEDs of emitter module 11 that undergo double modulation can be replaced by other light-emitting components, in particular laser diodes, to obtain different effects, in particular on germination of seeds or seedlings.

The fields of application of the present photo-bio-stimulation technology (i.e. the use of nanometric frequencies chopped and modulated by an LF and RF frequency) are as follows:
stimulation of cell responses in biological media (prokaryotic cells, eukaryotic cells, bacteria, yeasts, moulds . . . ),
treatment of cells in identical fields to those of phototherapy and photopheresis,
improvement of techniques used in cell biotechnologies (poration, transfection),
cell growth (cultures),
optimization of operation of nutritive media,
growth of embryos, hatching of eggs,
regeneration of bone fractures, skin, cartilages, tendons, muscles, repairing sectionalized nervous structures,
healing wounds,
treatment of arthritis and other inflammations,
veterinary field applications,
seedlings and plant growth,
action in gaseous media (air, oxygen etc., or other gas mixtures), in liquid medium, or on any living masses,
bio-reactors,
application in the water and sludge treatment and sewage fields.

Such a device enables certain cells and organisms to be treated selectively or specifically (mould cells, yeasts, fungi, algae, bacteria, plants, animal or human cells). The cells can be in a liquid medium in batch or in a fluid in circulation. Permeabilization of the cell membrane enables a protein or a chemical molecule to penetrate into the cell in a soft, non-aggressive manner. When the cell pores are open, transfers of exogenous molecules into the cells and of endogenous metabolites out of the cell can take place, the cells thereby being able to lose a large part of their ATP while remaining alive.

Depending on the parameters used, the device enables the cells and organisms to be destroyed or fosters cellular developments.

The present technology paves the way for new therapeutic applications, such as transfection of genes at nucleus level or induction of secondary cellular mechanisms implementing the artillery of cell signals. Such a device also facilitates assimilation in the case where functions are deficient.

1. An irradiation device by modulated photonic energy radiation comprising a nanometric-frequency photonic emitter module, wherein the emitter module is connected to modulating means performing a first chopping at low frequency, and a second chopping at radiofrequency to obtain a series of low frequency pulses chopped at radiofrequency.

2. The irradiation device according to claim 1, wherein the frequency range of the first chopping at low frequency is comprised between 5 Hz and 50 Hz, and the frequency range of the second chopping at radiofrequency is comprised between 500 kHz and 10,000 kHz.

3. The irradiation device according to claim 2, wherein the frequency range of the second chopping at radiofrequency is comprised between 1,000 kHz and 1,500 kHz.

4. The irradiation device according to claim 2, wherein the frequency range of the first chopping at low frequency is comprised between 10 Hz and 20 Hz.

5. The irradiation device according to claim 1, wherein the photonic emitter module, of predefined wavelength, is connected to a DC source by a transistor to form a chopping circuit.

6. The irradiation device according to claim 5, wherein the chopping circuit comprises a logic AND circuit to the inputs whereof a first oscillator at low frequency and a second oscillator at radio frequency (RF) are connected, and that the output of the logic circuit is connected to the transistor gate to modulate the power supply with double chopping by the two frequencies of said first and second oscillators.

7. The irradiation device according to claim 1, wherein the emitter module is equipped with a series of light-emitting diodes or laser diodes, with radiation near the infrared.

8. An irradiation method using an irradiation device comprising:
an application of nanometric-frequency photon emission to cells, chopping of the nanometric emission by low frequency, and chopping of the nanometric emission by radiofrequency.

9. The irradiation method according to claim 8 wherein the frequency range of the chopping at low frequency is comprised between 5 Hz and 50 Hz, and the frequency range of the chopping at radiofrequency is comprised between 500 kHz and 10,000 kHz.

10. The irradiation method according to claim 8, wherein the frequency range of the chopping at radiofrequency is comprised between 1,000 kHz and 1,500 kHz.

11. The irradiation method according to claim 8, wherein the frequency range of the chopping at low frequency is comprised between 10 Hz and 20 Hz.

12. The irradiation method according to claim 8, wherein cells are selected from a group consisting of mould, yeasts, fungi, algae, bacteria or plants and are treated selectively or specifically.

13. The irradiation method according to claim 8, wherein cells, isolated or forming a living substrate as organs, tissues, bones, muscles, tendons, are selected from a group consisting of animal or human cells and are treated selectively or specifically.

14. The irradiation method according to claim 8, wherein cellular activity is stimulated.

15. The irradiation method according to claim 8, wherein plant seedling, germination, growth and culture are stimulated.

16. The irradiation method according to claim 8, wherein cellular stress of cells is initiated.

17. The irradiation method according to claim 8, wherein cellular activity of cells is stimulated after freezing and thawing.

18. The irradiation method according to claim 8, wherein muscular pain, sprains, backache, and post-traumatic pains are relieved.

19. The irradiation method according to claim 8, wherein cells are in a liquid medium, in batch or in a fluid in circulation.

20. The irradiation method according to claim 8, wherein poration or transfection of cells is triggered.

21. The irradiation method according to claim 8, wherein cells are placed in a bioreactor.
